(19) United States
(12) Patent Application Publication
(10) Pub. No.: US 2024/0018520 A1
(43) Pub. Date: Jan. 18, 2024

(54) SRNA PLATFORM FOR INHIBITING PROKARYOTIC EXPRESSION AND USE THEREOF

(71) Applicant: KOREA ADVANCED INSTITUTE OF SCIENCE AND TECHNOLOGY, Daejeon (KR)

(72) Inventors: Sang Yup LEE, Daejeon (KR); Dongsoo YANG, Daejeon (KR); Jae Sung CHO, Daejeon (KR)

(21) Appl. No.: 18/340,838

(22) Filed: Jun. 24, 2023

Related U.S. Application Data
(63) Continuation-in-part of application No. 16/960,064, filed on Jul. 3, 2020, now abandoned.

(30) Foreign Application Priority Data
Mar. 8, 2018 (KR) 10-2018-0027544
Mar. 7, 2019 (KR) 10-2019-0026219

(51) Int. Cl. C12N 15/113; C12N 15/63

(52) U.S. Cl. CPC C12N 15/113; C12N 2310/10; C12N 15/63

(57) ABSTRACT

Synthetic sRNA for inhibiting prokaryote gene expression is described, which includes (i) an Hfq binding site and (ii) a region that forms a complementary bond with a target gene mRNA. Vectors encoding the synthetic sRNA are described, as well as recombinant prokaryotes transformed with such vectors, methods of inhibiting prokaryote gene expression, and methods of gene screening and strain improvement. The synthetic sRNA is able to control single and multiple target genes at a time, and is particularly useful for inhibiting a gene expression of Gram-positive bacteria, e.g., in a recombinant Corynebacterium for mass production of high value products that do not require fossil fuels with associated environmental problems.

### CROSS-REFERENCE TO RELATED APPLICATIONS

This is a continuation-in-part under 35 U.S.C. § 120 of U.S. patent application Ser. No. 16/960,064 filed Jul. 3, 2020, which in turn is a U.S. national phase under 35 U.S.C. § 371 of International Patent Application No. PCT/KR19/02715 filed Mar. 8, 2019, which in turn claims the priority under 35 U.S.C. § 119 of Korean Patent Application No. 10-2018-0027544 filed Mar. 8, 2018 and the priority under 35 U.S.C. § 119 of Korean Patent Application 10-2019-0026219 filed Mar. 7, 2019. The disclosures of all such applications are hereby incorporated herein by reference in their respective entireties, for all purposes.

### SEQUENCE LISTING

The application includes an electronically submitted sequence listing in .XML format. The .XML file contains a sequence listing entitled “523CIP_SegListing.xml created on Jun. 18, 2023 and is 55,931 bytes in size. The sequence listing contained in this .XML file is part of the specification and is hereby incorporated by reference herein in its entirety.

### TECHNICAL FIELD

The present disclosure relates to a novel sRNA platform for inhibiting prokaryotic expression and the use thereof, and more particularly to an sRNA platform for inhibiting prokaryotic expression, including synthetic sRNA including (i) an Hfq binding site derived from sRNA of any one selected from the group consisting of sprX2, roxS, arnA, surA, ASdes, ASpks, AS1726, AS1890, Mcr1˜19, Mpr1˜21, B11, B55, C8, F6, G2, ncRv12659, fsrA, crcZ, SR1, 6S-1, ncr1175, ncr982, ncr1241, ncr1015, ncr1241, ncr1575, ncr952, ncr629, cgb_03605, cgb_00105, cgb_20715, IGR-1˜12, AS-1˜12, sgs2672, sgs3323, sgs3618, sgs4453, sgs4827, sgs2746, sgs3903, sgs4581, sgs5362, sgs5676, sgs6100, sgs6109, scr1906, scr2101, scr3261, scr3261, a3287, scr3558, scr3974, scr4677 and scr5676, and (ii) a region forming a complementary bond with a target gene mRNA, and sRNA platform for inhibiting prokaryotic expression comprising a prokaryote-derived Hfq, a method of preparing the same and the use thereof.

### BACKGROUND ART

The prokaryote, Corynebacterium is a Gram-positive bacterium that is aerobic and non-pathogenic, has a short rod shape and does not form spores. Corynebacterium has a relatively small genome of about 3,309 kb. Corynebacterium has mainly been used as an industrial microorganism for the production of amino acids and nucleic acids by a microbial fermentation method since it was first isolated. Corynebacterium has several advantages that enable widespread use thereof as a production strain, in addition to the production of large amounts of amino acid and nucleic acid substances.

First, these strains do not produce toxic substances and thus pose relatively little danger to humans and livestock. Second, these strains have a relatively simple metabolic process and do not have multiplicity of enzymes observed in other microorganisms such as E. coli, thus having a simpler biosynthetic pathway regulation behavior than that of other microorganisms such as E. coli. Third, they cause almost no loss or leakage of proteins, thus providing high production efficiency during industrial production of amino acids and nucleic acids. Recent advances in gene recombination technologies such as gene cloning, gene amplification and gene inactivation have brought about the molecular genetic study of metabolic pathways of amino acids and nucleic acids in Corynebacterium, and these technologies have led to analysis and manipulation of metabolic pathways.

Construction of an eco-friendly and renewable biomass-based production system can be achieved by optimizing the metabolic flow for production of the target material through control of metabolic pathways in organisms using various molecular biology techniques. First, there are methods of increasing the expression of enzymes related to a target substance in order to improve metabolic flow required for the production of the target substance and of inducing deletion of a related gene in order to prevent metabolic flow competing with the target substance and cell growth. The method of deletion of the gene, which is one of current methods for regulating the metabolism of Gram-positive bacteria, includes a method of replacing any sequence having a homologous sequence with the target gene to be deleted through a recombination method and then inserting an antibiotic sequence into the target gene sequence, and a method of producing bacteria, the function of which is lost, through a second selection process using the SacB gene for the production of a strain from which the antibiotic resistance gene has been removed (Schafer, A et al., Gene, 145 (1), 69-73, 1994). However, such a gene deletion method has the following problems.

First, this method takes a longer time for gene deletion than other methods. Considering the time required for the first screening to replace the chromosomal gene using the homologous sequence and the time required for the second screening to screen the strain, from which the antibiotic resistance gene has been removed, using the SacB gene, it takes about three weeks or more to delete one gene. This is a factor that delays the efficient metabolic production of the target substance in bacteria.

Second, the method of removing the activity of the gene by inserting the antibiotic resistance gene into the chromosome to be deleted has a limitation with regard to the number of genes that can be deleted because the number of antibiotics that can be inserted into the chromosome is limited.

Third, it is difficult to recover a gene deleted from the chromosome manipulated by a conventional gene deletion method. In addition, when the same gene deletion is attempted in a different target strain, the overall process must be attempted again from the beginning, thus taking a lot of time and effort.

Therefore, in order to overcome the limitations pertaining to the above gene deletion, there is a need for methods that can reduce the expression of the target gene without changing the sequence of the Corynebacterium chromosome, control the degree of gene expression and easily apply the same gene expression control function to Gram-positive bacteria other than Corynebacterium.

Meanwhile, a gene expression inhibition system using synthetic sRNA in gram-negative bacteria such as E. coli has been developed by the present inventors in order to solve the above problems (KR 10-1575587, U.S. Pat. No. 9,388,417, Na, D et al., Nat. Biotechnol., 31 (2), 170-174, 2013; Yoo, S M et al. Nat. Protoc., 8(9), 1694-1707, 2013). The present inventors effectively suppressed gene expression in E. coli using the system, and developed a strain having increased production of cadaverine and tyrosine using the same (KR 10-1575587, U.S. Pat. No. 9,388,417, Na, D. et al., Nat. Biotechnol., 31 (2), 170-174, 2013; Yoo, S. M. et al. Nat. Protoc., 8 (9), 1694-1707, 2013), and further developed a strain having increased production of putrescine and proline in E. coli using the sRNA platform having various degrees of expression inhibition ability by changing the strength of the promoter expressing sRNA (KR 10-2015-0142304 A, KR 10-1750855 B1, Noh, M. et al., Cell Systems, 5, 1-9, 2017). In addition, the present inventors developed a strain with increased production of butanol by applying the system to microorganisms of the genus Clostridium (KR 10-2015-0142305 A, KR 10-1690780 B1, Cho, C. et al., 114 (2), 374-383, 2017).

Accordingly, as a result of intense efforts to solve these problems, the present inventors found that the expression of a target gene can be effectively inhibited by simultaneously expressing, in a prokaryote, synthetic sRNA including (i) an Hfq binding site derived from sRNA of any one selected from the group consisting of sprX2, roxS, arnA, surA, ASdes, ASpks, AS1726, AS1890, Mcr1˜19, Mpr1˜21, B11, B55, C8, F6, G2, ncRv12659, fsrA, crcZ, SR1, 6S-1, ncr1175, ncr982, ncr1241, ncr1015, ncr1241, ncr1575, ncr952, ncr629, cgb_03605, cgb_00105, cgb_20715, IGR-1˜12, AS-1˜12, sgs2672, sgs3323, sgs3618, sgs4453, sgs4827, sgs2746, sgs3903, sgs4581, sgs5362, sgs5676, sgs6100, sgs6109, scr1906, scr2101, scr3261, scr3261, a3287, scr3558, scr3974, scr4677 and scr5676, and (ii) a region forming a complementary bond with a target gene mRNA; and an Hfq protein derived from a prokaryote recognizing the sRNA, and thus completed the present disclosure based on this finding.

The information disclosed in this Background section is provided only for enhancement of understanding of the background of the present disclosure, and therefore it may not include information that forms the prior art that is already obvious to those skilled in the art.

### DISCLOSURE

### Technical Problem

It is one object of the present disclosure to provide a composition for inhibiting gene expression including synthetic sRNA that can regulate gene expression while overcoming the limitations of conventional methods of conducting gene deletion in prokaryotes, a method of preparing the same and the use thereof.

### Technical Solution

In accordance with one aspect of the present disclosure, the above and other objects can be accomplished by the provision of synthetic sRNA for inhibiting gene expression in a prokaryote, the synthetic sRNA including (i) an Hfq binding site derived from sRNA of any one selected from the group consisting of sprX2, roxS, arnA, surA, ASdes, ASpks, AS1726, AS1890, Mcr1˜19, Mpr1˜21, B11, B55, C8, F6, G2, ncRv12659, fsrA, crcZ, SR1, 6S-1, ncr1175, ncr982, ncr1241, ncr1015, ncr1241, ncr1575, ncr952, ncr629, cgb_03605, cgb_00105, cgb_20715, IGR-1˜12, AS-1˜12, sgs2672, sgs3323, sgs3618, sgs4453, sgs4827, sgs2746, sgs3903, sgs4581, sgs5362, sgs5676, sgs6100, sgs6109, scr1906, scr2101, scr3261, scr3261, a3287, scr3558, scr3974, scr4677 and scr5676, and (ii) a region forming a complementary bond with a target gene mRNA.

In another aspect of the present disclosure, provided are a nucleic acid encoding the synthetic sRNA, an expression vector including the nucleic acid, and a recombinant prokaryote introduced with the expression vector or a replicable form of the nucleic acid.

In another aspect of the present disclosure, provided are an expression vector including a nucleic acid encoding the sRNA and prokaryote-derived Hfq, and a recombinant prokaryote introduced with the expression vector or a replicable form of the nucleic acid.

In another aspect of the present disclosure, provided is a method of inhibiting expression of a target gene in a prokaryote, including culturing the recombinant prokaryote to inhibit mRNA of the target gene.

In another aspect of the present disclosure, provided is a method of screening a gene targeted for deletion for production of a useful substance including:
(a) inhibiting expression of at least one of genes present in a target strain for producing the useful substance and participating in a biosynthetic pathway of the useful substance using the method of inhibiting expression of a target gene; and
(b) selecting the gene, expression of which is inhibited, as the gene targeted for deletion for the production of the useful substance when a production yield of the useful substance is improved due to the inhibition of expression.

In another aspect of the present disclosure, provided is a method of improving a strain for producing a useful substance including deleting a gene screened by the method or a combination of the screened gene to produce a recombinant strain.

In a further aspect of the present disclosure, a synthetic sRNA for inhibiting gene expression in a prokaryote is provided, the synthetic sRNA comprising:
(i) an Hfq binding site of roxS sRNA comprising the sequence of SEQ ID NO: 42 derived from Bacillus subtilis amplified by a primer of SEQ ID NO: 19 or 20, or an Hfq binding site of arnA sRNA comprising the sequence of SEQ ID NO: 43 derived from Corynebacterium glutamicum amplified by a primer of SEQ ID NO: 15 or 16; and
(ii) a region forming a complementary bond with a target gene mRNA.

The synthetic sRNA in specific embodiments of the disclosure may be constituted, wherein the region forming the complementary bond with the target gene mRNA entirely or partially forms a complementary bond with nucleic acid sequences corresponding to a start of a ribosome binding site of the target gene mRNA to an end of a gene-coding sequence.

In various embodiments of the present disclosure, the prokaryote is any one selected from the group consisting of Escherichia coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhizobium, Rhodobacter, Zymomonas, Bacillus, Staphylococcus, Lactococcus, Streptococcus, Lactobacillus, Clostridium, Corynebacterium, Streptomyces, Bifidobacterium and Cyclobacterium.

Various other aspects of the present disclosure relating to the disclosed synthetic sRNA for inhibiting gene expression in a prokaryote include:
a nucleic acid encoding the sRNA;
an expression vector comprising a nucleic acid encoding the sRNA;
a recombinant prokaryote transformed with the sRNA;
a recombinant prokaryote transformed with a nucleic acid encoding the sRNA;
a recombinant prokaryote transformed with the expression vector comprising a nucleic acid encoding the sRNA;
a nucleic acid comprising the nucleic acid encoding the sRNA and a nucleic acid encoding prokaryote-derived Hfq;
the above nucleic acid, wherein the prokaryote-derived Hfq is any one selected from the group consisting of Escherichia coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhizobium, Rhodobacter, Zymomonas, Bacillus, Staphylococcus, Lactococcus, Streptococcus, Lactobacillus, Clostridium, Corynebacterium, Streptomyces, Bifidobacterium and Cyclobacterium;
a recombinant prokaryote transformed with the above nucleic acid;
an expression vector comprising a nucleic acid encoding the sRNA and a nucleic acid encoding prokaryote-derived Hfq; and
a recombinant prokaryote transformed with such expression vector.

In a further aspect, the disclosure relates to a method of inhibiting expression of a target gene in a prokaryote comprising culturing a recombinant prokaryote transformed with an expression vector comprising a nucleic acid encoding the sRNA according to the disclosure and a nucleic acid encoding prokaryote-derived Hfq, to inhibit mRNA of the target gene.

Another aspect of the disclosure relates to a method of screening a gene targeted for deletion for production of a useful substance comprising:
(a) inhibiting expression of at least one of genes present in a target strain for producing the useful substance in a biosynthetic pathway of the useful substance, by the above-described method of inhibiting expression of a target gene in a prokaryote; and
(b) selecting the gene, expression of which is inhibited, as the gene targeted for deletion for the production of the useful substance when a production yield of the useful substance is improved due to the inhibition of expression.

A further aspect of the disclosure relates to a method of improving a strain for producing a useful substance comprising deleting (i) a gene screened by the above-described screening method, or (ii) a combination of genes including the screened gene, to produce a recombinant strain.

Other aspects of the disclosure will be more fully apparent from the ensuing disclosure and appended claims.

### DESCRIPTION OF THE DRAWINGS

FIG. 1 in part a thereof shows a vector map of a cassette for inhibiting target gene expression according to an embodiment of the present disclosure, and in part b thereof shows the structure of a cassette for inhibiting target gene expression according to an embodiment of the present disclosure.

FIG. 2 shows the result of measurement of the expression inhibition ability of the target fluorescent protein in Corynebacterium when expressing various combinations of E. coli-derived synthetic regulatory sRNA, wherein Hfq represents E. coli Hfq, Hfqopt represents Corynebacterium codon-optimized Hfq, and antiGFP represents sRNA for inhibiting GFP target expression.

FIG. 3 shows the result of measurement of growth of strain Corynebacterium when expressing novel synthetic regulatory sRNA for prokaryotes.

FIG. 4 shows the result of measurement of the expression inhibition ability of a target fluorescent protein in Corynebacterium when expressing novel synthetic regulatory sRNA for prokaryotes.

FIG. 5 shows the result of measurement of the expression level of mRNA corresponding to the target fluorescent protein in Corynebacterium when expressing novel synthetic regulatory sRNA for prokaryotes.

FIG. 6 shows the result of measurement of a change in the expression level of the target gene (expression level of fluorescent protein) when the length of the target mRNA binding sequence of the synthetic regulatory sRNA is changed.

FIG. 7 shows the result of measurement of a change in lysine production when synthetic regulatory sRNA targeting the lysA gene is introduced into and expressed in a lysine-producing strain, namely the Corynebacterium BE strain.

FIG. 8 shows the result of measurement of a change in strain growth when synthetic regulatory sRNA targeting the pyc gene is introduced into and expressed in a wild-type Corynebacterium strain.

FIG. 9 shows the result of measurement of the change in the production of flaviolin when sRNA based on pEKEx1-bsuhfq-roxS platform targeting the rppA gene is introduced into E. coli expressing rppA (RppA+anti-rppA), wherein NC is a strain transformed with pEKEx1 as a wild-type E. coli control group.

### BEST MODE

Unless defined otherwise, all technical and scientific terms used herein have the same meanings as appreciated by those skilled in the field to which the present disclosure pertains. In general, the nomenclature used herein is well-known in the art and is ordinarily used.

Definitions of main terms used in the Detailed Description and the like of the present disclosure are as follows.

As used herein, the term “sRNA (small RNA)” refers to a short-length RNA typically having a base sequence length of 200 or less, which is not translated into a protein and effectively inhibits the translation of specific mRNA through complementary binding.

As used herein, the term “ribosome binding site” refers to a site of a ribosome binding to mRNA for transcription of mRNA.

As used herein, the term “gene” may encode a structural protein or regulatory protein. At this time, the regulatory protein includes a protein involved in a transcription factor, a heat shock protein, or DNA/RNA replication, transcription and/or translation. In the present disclosure, the gene targeted for inhibition of expression may be present as an extrachromosomal component.

In the present disclosure, the effect of inhibiting gene expression of novel synthetic regulatory sRNA that effectively acts in prokaryotes is confirmed.

In the present disclosure, sRNA including an Hfq binding site of sRNA derived from E. coli and a target gene mRNA-binding site, and sRNA including an Hfq binding site derived from Gram-positive bacteria and a target gene mRNA-binding site were produced, and the effect of inhibiting the expression of the target gene in Gram-positive bacteria thereof was confirmed.

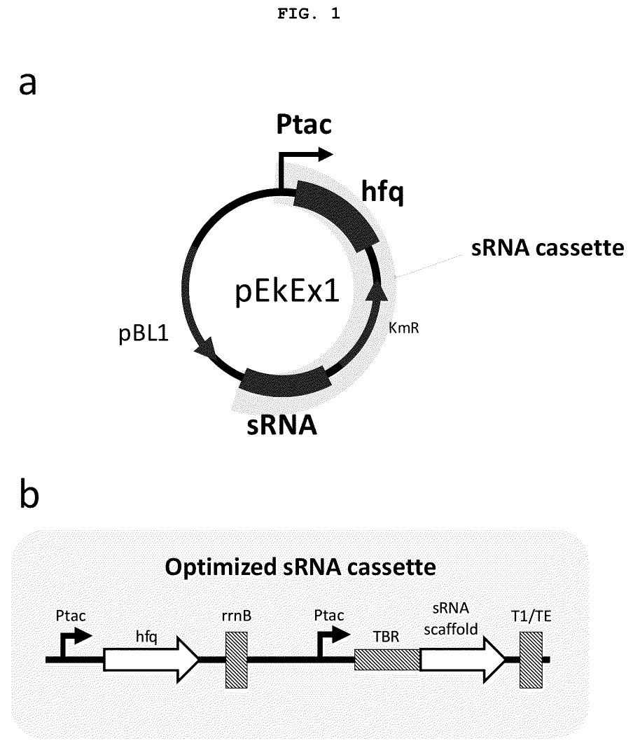

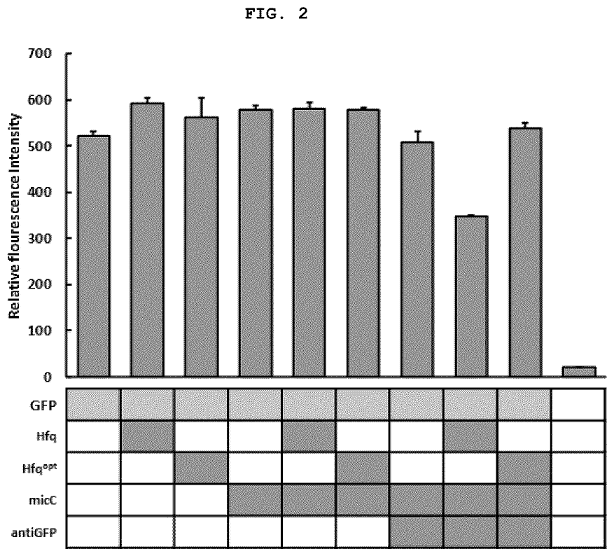

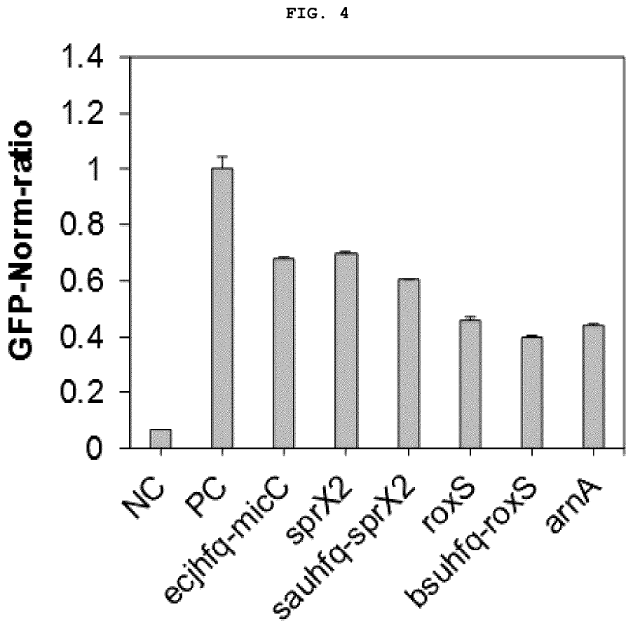

That is, in one embodiment of the present disclosure, sRNA including an Hfq-binding region extracted from sRNA derived from Escherichia coli, Bacillus subtilis, Staphylococcus aureus, or Corynebacterium glutamicum, and a region complementarily binding to the mRNA of an Hfq protein and GFP (green fluorescent protein) was produced (FIG. 1), and the effect of inhibiting gene expression was compared in Corynebacterium. The result showed that Gram-positive bacteria-derived synthetic regulatory sRNA exhibited a significantly higher expression inhibition efficiency in Corynebacterium than that of the conventional E. coli-derived synthetic regulatory sRNA (FIGS. 2 and 4).

In one aspect, the present disclosure is directed to synthetic sRNA for inhibiting gene expression in a prokaryote, the synthetic sRNA including (i) an Hfq binding site derived from sRNA of any one selected from the group consisting of sprX2, roxS, arnA, surA, ASdes, ASpks, AS1726, AS1890, Mcr1˜19, Mpr1˜21, B11, B55, C8, F6, G2, ncRv12659, fsrA, crcZ, SR1, 6S-1, ncr1175, ncr982, ncr1241, ncr1015, ncr1241, ncr1575, ncr952, ncr629, cgb_03605, cgb_00105, cgb_20715, IGR-1˜12, AS-1˜12, sgs2672, sgs3323, sgs3618, sgs4453, sgs4827, sgs2746, sgs3903, sgs4581, sgs5362, sgs5676, sgs6100, sgs6109, scr1906, scr2101, scr3261, scr3261, 0(3287, scr3558, scr3974, scr4677 and scr5676, and (ii) a region forming a complementary bond with a target gene mRNA.

In specific embodiments of the present disclosure, the synthetic sRNA comprises an Hfq binding site of roxS sRNA comprising the sequence of SEQ ID NO: 42 derived from Bacillus subtilis amplified by a primer of SEQ ID NO: 19 or 20, or an Hfq binding site of arnA sRNA comprising the sequence of SEQ ID NO: 43 derived from Corynebacterium glutamicum amplified by a primer of SEQ ID NO: 15 or 16.

roxS: 5′-acatatgaaaccgcgcttatcccggcgcggtttcttt-3′ (SEQ ID NO: 42)
arnA: 5′-aaaaaattacccctgaatagcctttaaaagactgttcgggggtaacgatgt-3′ (SEQ ID NO: 43)

In the present disclosure, the region forming the complementary bond with the target gene mRNA may entirely or partially form the complementary bond with a ribosome binding site of the target gene mRNA.

In the present disclosure, any type of prokaryote can be used as the prokaryote without limitation, and the prokaryote is preferably a Gram-positive bacteria or Gram-negative bacteria, more preferably a Gram-positive bacteria, but the present disclosure is not limited thereto.

In the present disclosure, the Gram-positive bacteria may be any one selected from the group consisting of Corynebacterium, Rhodococcus, Candida, Bacillus, Staphylococcus, Lactococcus, Streptococcus, Lactobacillus, Clostridium, Streptomyces and Bifidobacterium, but the present disclosure is not limited thereto.

In the present disclosure, the prokaryote may be any one selected from the group consisting of Escherichia coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhizobium, Rhodobacter, Zymomonas, Bacillus, Staphylococcus, Lactococcus, Streptococcus, Lactobacillus, Clostridium, Corynebacterium, Streptomyces, Bifidobacterium and Cyclobacterium.

The Hfq binding site derived from sRNA of any one selected from the group consisting of sprX2, roxS, arnA, surA, ASdes, ASpks, AS1726, AS1890, Mcr1˜19, Mpr1˜21, B11, B55, C8, F6, G2, ncRv12659, fsrA, crcZ, SR1, 6S-1, ncr1175, ncr982, ncr1241, ncr1015, ncr1241, ncr1575, ncr952, ncr629, cgb_03605, cgb_00105, cgb_20715, IGR-1˜12, AS-1˜12, sgs2672, sgs3323, sgs3618, sgs4453, sgs4827, sgs2746, sgs3903, sgs4581, sgs5362, sgs5676, sgs6100, sgs6109, scr1906, scr2101, scr3261, scr3261, a3287, scr3558, scr3974, scr4677 and scr5676 may be positioned continuously with the region forming the complementary bond with the target gene mRNA, or may be positioned apart therefrom via a linker such as a nucleic acid fragment.

Here, the term “complementary bond” refers to base pairing between nucleic acid sequences, and means that the sequence of a partial region of target gene mRNA and the sequence of the region forming a complementary bond with the target gene mRNA are complementary to each other by about 70-80% or more, preferably about 80-90% or more, even more preferably about 95-99% or more.

In addition, in the present disclosure, the sRNA of the present disclosure may be generally synthesized, but the present disclosure is not limited thereto.

That is, in the present disclosure, the sRNA may be chemically or enzymatically synthesized.

Accordingly, the sRNA according to the present disclosure may include a chemical modification. The chemical modification may be characterized in that the hydroxyl group at position 2′ of the ribose of at least one nucleotide included in the nucleic acid molecule is substituted with any one of a hydrogen atom, a fluorine atom, an —O-alkyl group, an —O-acyl group and an amino group, but the present disclosure is not limited thereto. In order to increase the transfer capacity of the nucleic acid molecule, the hydroxyl group may be substituted with any one of —Br, —Cl, —R, —R′OR, —SH, —SR, —N3 and —CN (R=alkyl, aryl, alkylene). In addition, the phosphate backbone of at least one nucleotide may be substituted with any one of a phosphorothioate form, a phosphorodithioate form, an alkylphosphonate form, a phosphoramidate form and a boranophosphate form. In addition, the chemical modification may be characterized in that at least one nucleotide included in the nucleic acid molecule is substituted with any one of LNA (locked nucleic acid), UNA (unlocked nucleic acid), morpholino, and PNA (peptide nucleic acid).

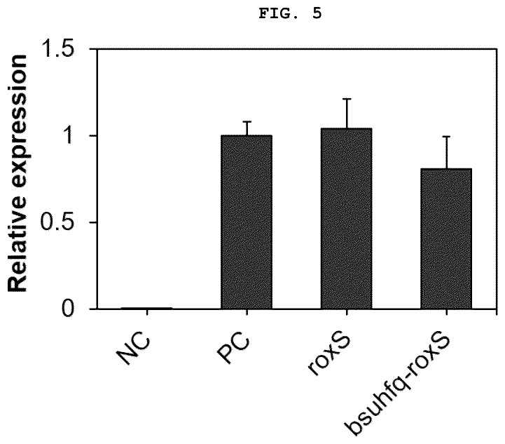

In another embodiment of the present disclosure, when the various synthetic regulatory sRNAs were introduced into Gram-positive bacteria in combination with a prokaryote-derived Hfq protein, the effect of inhibiting gene expression was found to be improved (FIGS. 4 and 5).

In another aspect, the present disclosure is directed to a nucleic acid encoding the sRNA, or the sRNA and prokaryote-derived Hfq, and an expression vector including the same.

In the present disclosure, the prokaryote may be any one selected from the group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhizobium, Rhodobacter, Zymomonas, Bacillus, Staphylococcus, Lactococcus, Streptococcus, Lactobacillus, Clostridium, Corynebacterium, Streptomyces, Bifidobacterium and Cyclobacterium.

In the present disclosure, the Hfq may be codon-optimized.

In the present disclosure, the term “nucleic acid” may refer to RNA, DNA, stabilized RNA or stabilized DNA. Here, “encoding” means encoding the sRNA, and encoded sRNA means a nucleic acid sequence complementary to the sRNA.

As used herein, the term “vector” means a DNA product containing a DNA sequence operably linked to a suitable control sequence capable of expressing DNA in a suitable host. The vector may be a plasmid, a phage particle or a simple potential genome insert. Once the vector is transformed into an appropriate host, it may replicate and function independently of the genome of the host, or may often be integrated with the genome. Since the plasmid is the most commonly used type of vector, the terms “plasmid” and “vector” may be used interchangeably throughout the specification of the present disclosure. For the purpose of the present disclosure, a plasmid vector is preferably used. A typical plasmid vector that can be used for this purpose includes (a) a replication origin to efficiently conduct replication so as to include several to several hundred plasmid vectors in each host cell, (b) an antibiotic resistance gene to screen a host cell transformed with the plasmid vector, and (C) a restriction enzyme cleavage site into which a foreign DNA fragment is inserted. Even if an appropriate restriction enzyme cleavage site is not present, the vector and foreign DNA can be easily ligated using a synthetic oligonucleotide adapter or a linker according to a conventional method. After ligation, the vector should be transformed into an appropriate host cell. Transformation can be easily carried out using a calcium chloride method or electroporation (Neumann, et al., EMBO J., 1: 841, 1982). As the vector used for the expression of sRNA according to the present disclosure, any expression vector known in the art may be used.

When a base sequence is aligned with a nucleic acid sequence based on a functional relationship, it is “operably linked” thereto. This may be gene(s) and control sequence(s) linked in such a way so as to enable gene expression when a suitable molecule (e.g., a transcriptional activator protein) is linked to the control sequence(s). For example, DNA for a pre-sequence or secretory leader is operably linked to DNA for a polypeptide, when expressed as a pre-protein involved in the secretion of the polypeptide; and a promoter or enhancer is operably linked to a coding sequence when it affects the transcription of the sequence; or a ribosome binding site is operably linked to a coding sequence when it affects the transcription of the sequence; or the ribosome binding site is operably linked to a coding sequence when positioned to facilitate translation. Generally, “operably linked” means that the linked DNA sequence is in contact therewith, or that a secretory leader is in contact therewith and is present in the reading frame. However, the enhancer need not be in contact therewith. The linkage of these sequences is carried out by ligation (linkage) at convenient restriction enzyme sites. When no such site exists, a synthetic oligonucleotide adapter or a linker according to a conventional method is used.

In another aspect, the present disclosure is directed to a nucleic acid encoding the synthetic sRNA, an expression vector including the nucleic acid, and a recombinant prokaryote introduced with the expression vector or a replicable form of the nucleic acid.

In another aspect, the present disclosure is directed to a nucleic acid encoding the synthetic sRNA, a nucleic acid encoding prokaryote-derived Hfq, an expression vector including each of the nucleic acids, and a recombinant prokaryote introduced with the expression vector or a replicable form of the nucleic acid.

In another aspect, the present disclosure is directed to an expression vector including the nucleic acid encoding the sRNA and prokaryote-derived Hfq, and a recombinant prokaryote introduced with the expression vector or a replicable form of the nucleic acid.

In the present disclosure, the prokaryote may be any one selected from the group consisting of E. coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhizobium, Rhodobacter, Zymomonas, Bacillus, Staphylococcus, Lactococcus, Streptococcus, Lactobacillus, Clostridium, Corynebacterium, Streptomyces, Bifidobacterium and Cyclobacterium.

As used herein, the term “transformation” means introducing DNA into a host and making the DNA replicable using an extrachromosomal factor or chromosomal integration.

It should be understood that not all vectors function identically in expressing the DNA sequences of the present disclosure. Similarly, not all hosts function identically in the same expression system. However, those skilled in the art will be able to make appropriate selections from a variety of vectors, expression control sequences and hosts without excessive burden of experimentation while not departing from the scope of the present disclosure. For example, selection of a vector should be carried out in consideration of the host, because the vector should be replicated therein. The number of replications of the vector, the ability to control the number of replications, and the expression of other proteins encoded by the corresponding vector, such as the expression of antibiotic markers, should also be considered.

In another aspect, the present disclosure is directed to a method of inhibiting expression of a target gene, including culturing the recombinant prokaryote to inhibit mRNA expression of the target gene.

At this time, preferably, the expression of the sRNA may be carried out using a promoter that acts in response to binding of an inducer such as arabinose or IPTG. That is, for tight expression of synthetic sRNA, sRNA expression can be regulated using an external inducer. In this case, specifically, the synthetic sRNA can be expressed using a tac promoter.

The sRNA and Hfq according to the present disclosure can be used to screen a target gene for production of a useful substance and the screening method may include (a) inhibiting expression of at least one of genes present in a target strain for producing the useful substance and participating in a biosynthetic pathway of the useful substance using the method of inhibiting expression of a target gene and (b) selecting the gene, expression of which is inhibited, as the gene targeted for deletion for the production of the useful substance when a production yield of the useful substance is improved due to the inhibition of expression.

As used herein, the term “deletion” includes inhibition of the activity of the corresponding enzyme by mutation, substitution, or deletion of some bases of the corresponding gene, introduction of some bases, or introduction of a gene, enzyme or chemical substance that inhibits the expression or activity of the corresponding enzyme. In addition, the gene targeted for deletion, screened as described above, can be used to improve a strain for producing a useful substance.

In another aspect of the present disclosure, provided is a method of improving a strain for producing a useful substance including deleting a gene screened by the method or a combination of the screened gene to produce a recombinant strain.

As used herein, the term “loss of function” includes inhibition of the activity of the corresponding enzyme by mutation, substitution, or deletion of some bases of the corresponding gene, introduction of some bases, or introduction of a gene, enzyme or chemical substance that inhibits the expression or activity of the corresponding enzyme. Therefore, the method of losing the function of a specific gene includes expression inhibition using known antisense RNA, homologous recombination, homologous recombination through expression of various recombinant enzymes (lambda recombinase, etc.), insertion of specific sequences using reverse transcriptase and RNA and the like, and is not limited to any particular method, as long as the activity of the specific target gene and the enzyme encoded by the gene are inhibited.

The present disclosure is also directed to a method of determining a target gene mRNA base-pairing region that forms a complementary bond with a target gene mRNA in consideration of all secondary structures of host mRNA in order to inhibit each gene in the most efficient and predictable manner, in light of the fact that the secondary structure of the mRNA of the target gene affects inhibition of gene expression.

In the present disclosure, the method includes: (a) inputting a target gene and a host strain; (b) obtaining an RNA sequence including transcription starting points of all genes of a host strain; and (c) determining a sequence of a region complementarily binding to a target gene of sRNA according to conditions.

In the present disclosure, the conditions may be characterized in that the specific sRNA does not perform non specific interaction (off-targeting) with a gene having a different genome phase excluding the target gene.

### EXAMPLES

Hereinafter, the present disclosure will be described in more detail with reference to examples. However, it will be obvious to those skilled in the art that these examples are provided only for illustration of the present disclosure and should not be construed as limiting the scope of the present disclosure.

### Example 1: Confirmation of Performance of E. coli-Derived Synthetic Regulatory sRNA

A Corynebacterium strain was used as a representative Gram-positive bacterium, and pEKEx1 (Eikmanns et al., Gene 102 (1): 93-98, 1991), a vector frequently used in the corresponding strain, was used as a platform vector (FIG. 1). First, the effects of several factors including Hfq, MicC and anti-GFP sequences, which are conventional E. coli sRNA systems, on the expression inhibition of sRNA were determined. That is, various sRNA expression vectors were constructed depending on the presence or absence of Hfq derived from E. coli W3110, the presence or absence of the codon optimization of Hfq for expression in Corynebacterium, and the presence or absence of an anti-GFP sequence, and were expressed along with GFP expressed through the I16 synthetic promoter on pCES208 (Yim S. S., et al., Biotechnol. Bioeng., 110: 2959, 2013), and the gene expression inhibitory ability thereof was confirmed.

The characteristics of the vectors developed in the present disclosure are shown in Table 1 below.

TABLE 1
Vectors for E. coli sRNA system test

| Vector name | Characteristics |
|---|---|
| pEKEx1-ecjhfq | Including E. Coli W3110-derived |
| | Hfq-encoding gene |
| pEKEx1-cglhfq | Including codon-optimized Hfq-encoding |
| | gene |
| pEKEx1-micC | Including Hfq-binding region of |
| | E. Coli-derived MicC sRNA |
| pEKEx1-ecjhfq-micC | Including both Hfq-encoding gene and |
| | MicC Hfq-binding region |
| pEKEx1-cglhfq-micC | Including codon-optimized Hfq-encoding |
| | gene and MicC Hfq-binding region |
| pEKEx1-micC_antiGFP | Including both MicC Hfq binding region |
| | and GFP mRNA-binding sequence |
| pEKEx1-ecjhfq-micC- | Including Hfq-encoding gene, MicC |
| antiGFP | Hfq-binding region GFP target |
| | gene-binding region |
| pEKEx1-cglhfq-micC- | Including codon-optimized Hfq-encoding |
| antiGFP | gene, MicC Hfq-binding region and GFP |
| | target gene-binding region |

First, in order to introduce E. coli-derived hfq into the pEKEx1 vector, the pEKEx1 vector was treated with EcoRI and PstI restriction enzymes, and then the pEKEx1 vector treated with EcoRI and PstI restriction enzymes was assembled through Gibson assembly with the DNA fragment obtained through PCR using the primers of [SEQ ID NO: 1] and [SEQ ID NO: 2] and using the genome of E. coli W3110 as a template to produce a pEKEx1-ecjhfq vector. Here, the sequence of the E. coli-derived hfq is shown in [SEQ ID NO: 3].

Similarly, after E. coli-derived hfq was Corynebacterium codon-optimized (synthesized by Bioneer Corp.), the same procedure as above was performed to introduce the result into the pEKEx1 vector. At this time, the sequences of the primers used for PCR are the same as [SEQ ID NO: 4] and [SEQ ID NO: 5]. The codon-optimized Hfq encoding gene produced through PCR was designated as “cg1hfq”. The vector thus produced was designated as “pEKEx1-cg1hfq”. The sequence of cg1hfq is shown in SEQ ID NO: 28 below.

[SEQ ID NO: 1]
5′-caatttcacacaggaaacagaattcATGGCTAAGGGGCAATCTTTA
C-3′
[SEQ ID NO: 2]
5′-caccatatctatatctccttgaattcATTATTCGGTTTCTTCGCTGT
CC-3′
[SEQ ID NO: 3]
5′-atggctaaggggcaatctttacaagatccgttcctgaacgcactgcg
tcgggaacgtgttccagtttctatttatttggtgaatggtattaagctgc
aagggcaaatcgagtcttttgatcagttcgtgatcctgttgaaaaacacg
gtcagccagatggtttacaagcacgcgatttctactgttgtcccgtctcg
cccggtttctcatcacagtaacaacgccggtggcggtaccagcagtaact
accatcatggtagcagcgcgcagaatacttccgcgcaacaggacagcgaa
gaaaccgaataa-3′
[SEQ ID NO: 4]
5′-caatttcacacaggaaacagaattcATGGCTAAGGGTCAGTCTCTC-
3′
[SEQ ID NO: 5]
5′-caccatatctatatctccttgaattcATTACTATTCGGTTTCCTCG
G-3′
[SEQ ID NO: 28]
5′-atggctaagggtcagtctctccaggacccattcttgaacgcactgcg
tcgcgaacgcgtgcccgtgtccatctatctggtgaacggtattaaacttc
agggacagatcgagtccttcgatcagtttgttatcctgctcaagaacacg
gtctcccagatggtatacaagcatgcgatttcaaccgttgtcccttcccg
cccggtgtctcaccactcgaacaatgccggcggcggcacctcctccaact
accaccacggcagcagcgcccaaaacacttccgcacagcaggattccgag
gaaaccgaatagtaa-3′

After introducing the hfq as above, for introduction of a MicC-based sRNA platform, E. coli-derived hfq (ecjhfq), Corynebacterium codon-optimized hfq (cg1hfq), or the pEKEx1 vector not inserted with hfq was treated with the StuI restriction enzyme, and then PCR amplification was conducted using the E. coli W3110 genome as a template, and using primers [SEQ ID NO: 6] and [SEQ ID NO: 7] to produce a first sRNA fragment, and the first sRNA fragment was amplified again by PCR using primers [SEQ ID NO: 8] and [SEQ ID NO: 9] to produce a micC sRNA fragment. The DNA fragment thus formed was assembled with the pEKEx1-based vectors treated with the StuI using Gibson assembly to produce a pEKEx1-micC vector. At this time, pEKEx1-ecjhfq and pEKEx1-cg1hfq vectors were treated with StuI in the same manner as above, and then the micC sRNA fragment was assembled thereto using Gibson assembly to produce pEKEx1-ecjhfq-micC and pEKEx1-cg1hfq-micC vectors.

In addition, the following experiment was performed to produce an sRNA vector including a target mRNA-binding sequence for inhibition of the expression of a GFP fluorescent protein. First, the first sRNA fragment was amplified by PCR using the E. coli W3110 genome as a template and using primers [SEQ ID NO: 10] and [SEQ ID NO: 7], and was amplified again by PCR using primers [SEQ ID NO: 8] and [SEQ ID NO: 9]. The resulting micC-antiGFP sRNA fragment was assembled to the pEKEx1-micC vector, the pEKEx1-ecjhfq-micC vector and the pEKEx1-cg1hfq-micC vector, each treated with StuI restriction enzyme, using Gibson assembly, to produce pEKEx1-micC-antiGFP, pEKEx1-ecjhfq-micC-antiGFP and pEKEx1-cg1hfq-micC-antiGFP vectors, respectively.

[SEQ ID NO: 6]
5′-ttgacaattaatcatcggctcgtataatgtgtggAGCTCTCATTTTG
CAGATTTgttttagagctagaaatagcaagt-3′
[SEQ ID NO: 7]
5′-TATAGATATCCCGCGGTATATTAATTAATATAAACGCAGAAAGGCC
C-3′
[SEQ ID NO: 8]
5′-TGGATGATGGGGCGATTCAGGtatagatatcTTGACAATTAATCATC
GGCT-3′
[SEQ ID NO: 9]
5′-AAGGTGTTGCTGACTCATACCAGGTATAGATATCCCGCGGTATA-3′
[SEQ ID NO: 10]
5′-ttgacaattaatcatcggctcgtataatgtgtggGAAAAGTTCTTCT
CCTTTACTCATtttctgttgggccattgcattg-3′

For the construction of a pCES208-I16-GFP vector for use as a reporter plasmid, substitution of the GFP expression vector constructed through the conventional studies with a spectinomycin marker for efficient use was conducted before use thereof (Yim, S. S., Biotechnol. Bioeng., 110(11), 2959-2969, 2013).

Each of the vectors constructed as above was transformed into a strain of Corynebacterium, and then culturing was conducted. The culture method is as follows. First, pCES208-I16-GFP was transformed and then screened in BHIS plate medium (37 g/L of brain heart infusion (BHI), 91 g/L of sorbitol, 15 g/L of agar) supplemented with 200 μg/L of spectinomycin.

The sRNA vector of Table 1 was introduced into a strain capable of expressing the fluorescent protein, and was then screened again in a BHIS plate medium supplemented with both kanamycin and spectinomycin. A total of 8 strains including the screened 7 recombinant strains and the wild-type ATCC13032 strain were inoculated in a test tube containing 2 mL of BHIS medium (37 g/L of brain heart infusion (BHI), 91 g/L of sorbitol), and then pre-incubated at 30° C. for 16 hours. The pre-cultured culture solution was inoculated in an amount enabling the OD₆₀₀ to be 0.1 in the next BHIS 2 ml test tube, and simultaneously 1 mM of IPTG was added thereto, followed by culturing for 24 hours.

After the culture, OD (optical density) was measured at a wavelength of 600 nm in order to measure the growth of cells, and additionally, some of the cells were washed twice with phosphate-buffered saline (PBS) and isolated in 1 mL of PBS, and fluorescence protein expression was measured by FACS (fluorescence-activated cell sorting).

As shown in FIG. 2, the result showed that gene expression inhibition hardly occurred in all other platforms, whereas, when E. coli-derived Hfq, MicC and anti-GFP were simultaneously expressed, GFP expression in Corynebacterium was inhibited at an efficiency of about 35%. This is lower than the target gene expression inhibition ability as identified in the conventional literature (D. Na et al., Nat. Biotechnol. (2013), 31(2), 170) or the patent (KR 10-1575587-0000). Thus, construction of a novel sRNA expression platform was required.

### Example 2: Construction of Novel sRNA Platform Derived from Gram-Positive Bacteria

A vector was constructed in the same manner as in Example 1, except that the types of Hfq protein and sRNA Hfq binding sites were changed to those derived from Gram-positive bacteria.

Respective Hfq proteins and sRNAs are shown in Table 2 below.

TABLE 2
Novel sRNA components

| Name | Characteristics |
|---|---|
| bsuhfq | Bacillus subtilis-derived hfq |
| sauhfq | Staphylococcus aureus-derived hfq |
| roxS | Bacillus subtilis-derived sRNA scaffold |
| sprX2 | Staphylococcus aureus-derived sRNA scaffold |
| arnA | Corynebacterium glutamicum-derived sRNA scaffold |

Table 3 below summarizes the vectors including respective components.

TABLE 3
New sRNA vector configuration

| Vector name | Characteristics |
|---|---|
| pEKEx1-sauhfq-sprX2 | Staphylococcus hfq + sRNA |
| pEKEx1-sauhfq | Staphylococcus hfq |
| pEKEx1-sprX2 | Staphylococcus sRNA |
| pEKEx1-bsuhfq-roxS | Bacillus hfq + sRNA |
| pEKEx1-bsuhfq | Bacillus hfq |
| pEKEx1-roxS | Bacillus sRNA |
| pEKEx1-arnA | Corynebacterium SRNA |

In order to construct the sRNA platform vectors, first, each hfq was inserted into the pEKEx1 vector. For this purpose, the pEKEx1 vector was treated with EcoRI and PstI restriction enzymes, and then the bsuhfq DNA fragment was amplified by PCR using the primers [SEQ ID NO: 11] and [SEQ ID NO: 12] and using the Bacillus subtilis genome as a template, and similarly, the sauhfq DNA fragment was amplified by PCR using the primers [SEQ ID NO: 13] and [SEQ ID NO: 14] and using the Staphylococcus aureus genome as a template. The DNA fragments thus amplified were assembled to the vector treated with the restriction enzyme using Gibson assembly.

[SEQ ID NO: 11]
5′-caatttcacacaggaaacagaattcATGAAACCGATTAATATTCAG-
3′
[SEQ ID NO: 12]
5′-caaaacagccaagcttggctgcagATTATTCGAGTTCAAGCTGGAC-
3′
[SEQ ID NO: 13]
5′-CAATTTCACACAGGAAACAGAATTCATGATTGCAAACGAAAACATC-
3′
[SEQ ID NO: 14]
5′-CAAAACAGCCAAGCTTGGCTGATTATTCTTCACTTTCAGTAG-3′

Then, each sRNA scaffold was inserted into a pWAS vector (KR 10-1575587), which is a conventional sRNA platform vector for expression of E. coli. At this time, an inverse PCR technique was performed using the pWAS vector as a template. At this time, primers [SEQ ID NO: 15] and [SEQ ID NO: 16] were used for the construction of arnA, primers [SEQ ID NO: 17] and [SEQ ID NO: 18] were used for the construction of sprX2, and primers [SEQ ID NO: 19] and [SEQ ID NO: 20] were used for the construction of roxS.

Inverse PCR was performed using an unmanipulated pWAS vector as a template and using the primers, and only the DNA fragment amplified through the DpnI restriction enzyme was left from respective DNA fragments and the template that was originally inserted was removed, and phosphoric acid groups were attached to both ends of the DNA using T4 PNK (T4 polynucleotide kinase) to conduct ligation using T4 ligase. Respective sRNA scaffolds constructed on the pWAS vectors were used as templates in the subsequent PCR amplification experiments.

Then, a final sRNA vector for inhibiting GFP expression was constructed. PCR amplification was conducted using each pWAS-based vector as a template and using primers [SEQ ID NO: 21] and [SEQ ID NO: 7] for arnA scaffold-based platforms, primers [SEQ ID NO: 22], [SEQ ID NO: 7] for sprX2 scaffold-based platforms, and primers [SEQ ID NO: 23] and [SEQ ID NO: 7] for roxS-based platforms. The amplified arnA sRNA fragment was assembled with the pEKEx1 vector treated with the StuI restriction enzyme to produce a pEKEx1-arnA vector. In addition, the spEX2 sRNA fragment was assembled with the pEKEx1 vector and with the pEKEx1-sauhfq vector, each treated with the StuI restriction enzyme, to produce pEKEx1-sprX2 and pEKEx1-sauhfq-sprX2 vectors, respectively. The final roxS sRNA fragment was assembled with the pEKEx1 vector and the pEKEx1-bsuhfq vector, each treated with StuI restriction enzyme, to produce pEKEx1-roxS and pEKEx1-bsuhfq-roxS vectors, respectively. At this time, each amplified DNA fragment was subsequently amplified again using [SEQ ID NO: 8] and [SEQ ID NO: 9], and then assembled with the pEKEx1-based hfq expression vector treated with StuI restriction enzyme using Gibson assembly (FIG. 1).

As shown in FIG. 1 in part a thereof, the result showed that a sRNA expression cassette having a structure of a promoter (Ptac)-gram-positive bacteria-derived hfq-coding gene-terminator (rrnB) and a promoter (Ptac)-target mRNA binding region (TBR)-sRNA scaffold-terminator (T1/TE) was produced.

[SEQ ID NO: 15]
5′-aaaggctattcaggggtaattttttCGGGGGATCCACTAGTTCTAG-
3′
[SEQ ID NO: 16]
5′-aaaagactgttcgggggtaacgatgtCTCGAGCCAGGCATCAAATAA
AAC-3′
[SEQ ID NO: 17]
5′-acccagtgacatgcttgggtgaCGGGGGATCCACTAGTTCTAG-3′
[SEQ ID NO: 18]
5′-gttttttcttacgatagagagcaCTCGAGCCAGGCATCAAATAAAA
C-3′
[SEQ ID NO: 19]
5′-aagcgcggtttcatatgtCGGGGGATCCACTAGTTCTAG-3′
[SEQ ID NO: 20]
5′-atcccggcgcggtttctttCTCGAGCCAGGCATCAAATAAAAC-3′
[SEQ ID NO: 21]
5′-ttgacaattaatcatcggctcgtataatgtgtggGAAAAGTTCTTCT
CCTTTACTCATAAAAAATTACCCCTGAATAGCC-3′
[SEQ ID NO: 22]
5′-ttgacaattaatcatcggctcgtataatgtgtggGAAAAGTTCTTCT
CCTTTACTCATTCACCCAAGCATGTCACTGG-3′
[SEQ ID NO: 23]
5′-ttgacaattaatcatcggctcgtataatgtgtggGAAAAGTTCTTCT
CCTTTACTCATACATATGAAACCGCGCTTATC-3′

### Example 3: Confirmation of Gene Expression Inhibition Ability of Constructed Platform

Each of the five types of novel sRNA platforms constructed in Example 2 and the conventional highest-efficiency E. coli-derived sRNA platform was transformed into a Corynebacterium strain containing pCES208-I16-GFP, and GFP fluorescence protein, mRNA expression and strain growth were then measured.

First, the sRNA platforms constructed in Example 2 were introduced into a strain capable of expressing a fluorescent protein and then were screened in a BHIS plate medium supplemented with both kanamycin and spectinomycin, and then a total of 8 strains including 7 recombinant strains and the wild-type ATCC13032 strain were inoculated into a test tube containing 2 mL of a BHIS medium (37 g/L of brain Heart Infusion (BHI), 91 g/L of sorbitol) and then pre-incubated at 30° C.; for 16 hours. The pre-culture solution was then inoculated in an amount enabling the OD₆₀₀ to be 0.1 in the next BHIS 2 ml test tube, and at the same time, 1 mM of IPTG was added, followed by culturing for 24 hours. After the culture, OD (optical density) was measured at a wavelength of 600 nm in order to measure the growth of cells, and additionally, some of the cells were washed twice with phosphate-buffered saline (PBS) and then isolated in 1 mL of PBS, and fluorescence protein expression was measured by FACS (fluorescence-activated cell sorting). In addition, RT-qPCR (reverse transcriptase-quantitative PCR) was performed to determine the change in expression level of the corresponding mRNA of the GFP gene depending on the presence of sRNA. At this time, primers [SEQ ID NO: 24] and [SEQ ID NO: 25] were used to amplify housekeeping mRNA, and primers [SEQ ID NO: 26] and [SEQ ID NO: 27] were used to amplify GPP mRNA.

| [SEQ ID NO: 24] | |
|---|---|
| | 5′-TGCACTACTGGAAAACTACC-3′ |
| [SEQ ID NO: 25] | |
| | 5′-TGTAGTTCCCGTCATCTTTG-3′ |
| [SEQ ID NO: 26] | |
| | 5′-GAAAACACCATCACCATTC-3′ |
| [SEQ ID NO: 27] | |
| | 5′-GTCTGGTAAACCAGGGACTC-3′ |

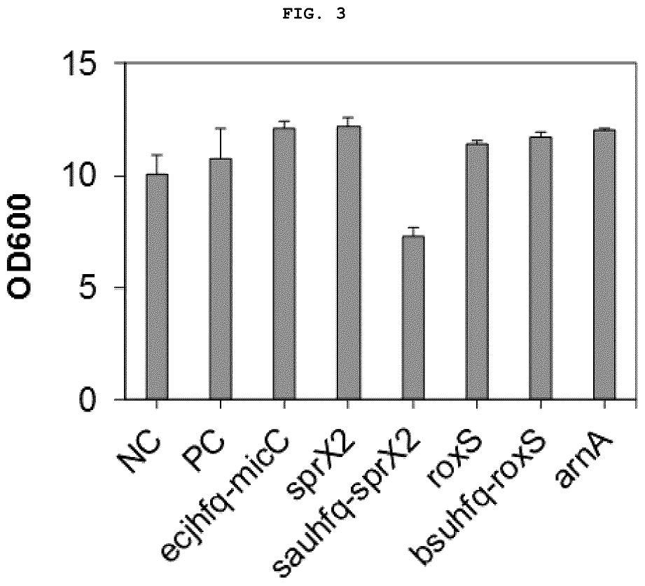

The result showed that, first, strains transformed using all platforms excluding the sauhfq-sprX2 platform exhibited growth similar to that of the control strain (FIG. 3, NC: Corynebacterium not introduced with a vector, PC: Recombinant Corynebacterium introduced only with GFP expression vector).

In addition, as shown in FIG. 4, the bsuhfq-roxS platform exhibits significantly higher inhibition ability on target gene expression when compared to the sRNA platform derived from E. coli of ecjhfq-micC, and as shown in FIG. 5, when roxS-based sRNA was expressed, regardless of the presence or absence of bsuhfq expression, significant fluorescence protein inhibition ability was detected while there was little inhibition of mRNA expression.

### Example 4: Testing for Effect of Various Lengths of Target mRNA Binding Sequences on Inhibition Ability of Target Gene Expression

In order to apply the sRNA-based target gene expression inhibition system (pEKEx1-bsuhfq-roxS) constructed in Example 3 under various conditions, the target mRNA binding sequence originally composed of a 24-bp base sequence was modified to various lengths, and then the inhibition ability of target gene expression was tested. The nucleotide sequence lengths tested by the present inventors were 16-bp to 40-bp, corresponding to the following [SEQ ID NO: 29] to [SEQ ID NO: 41], all of which are sequences from the initiation codon of the GFP fluorescent protein expression gene. However, it will be obvious to those skilled in the art that the available length of the target mRNA binding sequence is not limited to the length of the base sequence described above. Construction of the corresponding sRNA expression plasmids was performed in the same manner as in Example 2.

| [SEQ ID NO: 29] | |
|---|---|
| | 5′-atgagcaaaggagaag-3′ |
| [SEQ ID NO: 30] | |
| | 5′-atgagcaaaggagaagaa-3′ |
| [SEQ ID NO: 31] | |
| | 5′-atgagcaaaggagaagaact-3′ |
| [SEQ ID NO: 32] | |
| | 5′-atgagcaaaggagaagaacttt-3′ |
| [SEQ ID NO: 33] | |
| | 5′-atgagcaaaggagaagaacttttc-3′ |
| [SEQ ID NO: 34] | |
| | 5′-atgagcaaaggagaagaacttttcac-3′ |
| [SEQ ID NO: 35] | |
| | 5′-atgagcaaaggagaagaacttttcactg-3′ |
| [SEQ ID NO: 36] | |
| | 5′-atgagcaaaggagaagaacttttcactgga-3′ |
| [SEQ ID NO: 37] | |
| | 5′-atgagcaaaggagaagaacttttcactggagt-3′ |
| [SEQ ID NO: 38] | |
| | 5′-atgagcaaaggagaagaacttttcactggagttg-3′ |
| [SEQ ID NO: 39] | |
| | 5′-atgagcaaaggagaagaacttttcactggagttgtc-3′ |
| [SEQ ID NO: 40] | |
| | 5′-atgagcaaaggagaagaacttttcactggagttgtccc-3′ |
| [SEQ ID NO: 41] | |
| | 5′-atgagcaaaggagaagaacttttcactggagttgtcccaa-3′ |

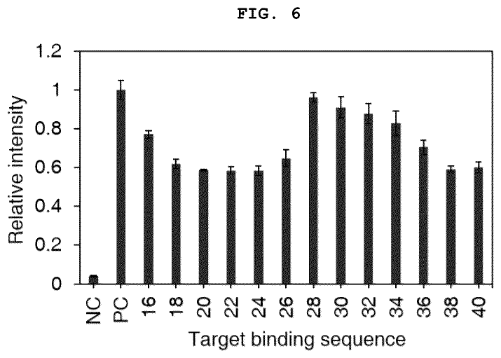

In order to test the ability of these synthetic regulatory sRNAs to inhibit target gene expression on the C. glutamicum genome, a gene encoding the sfGFP fluorescent protein was inserted between intrinsic genes bioD to be expressed under the H36 promoter. 13 sRNA expression plasmids (based on the pEKEx1-bsuhfq-roxS platform), having target mRNA binding sequences having different lengths, constructed as described above, were introduced into the constructed fluorescent protein expression strain, and the strains were inoculated into 2 mL of a BHIS medium (37 g/L of brain heart infusion (BHI), 91 g/L of sorbitol) supplemented with an antibiotic. The strains were cultured for 24 hours at 30° C. and 220 rpm, and passage-cultured for 24 hours under the same conditions. The results of measurement of the sfGFP fluorescent protein expression of the cultured strains are shown in FIG. 6.

As shown in FIG. 6, when the target mRNA binding sequence was 20, 22, or 24 bp, the expression inhibition ability was found to be the highest. Since high target specificity was also important, experiments were conducted using a length of 24 bp in the following examples.

### Example 5: Confirmation of Expression Inhibition Ability of Target Gene on Corynebacterium Genome

5-1. Confirmation of Inhibition Ability of lysA Gene Expression

The expression inhibition ability for the target gene on the Corynebacterium genome was tested using the sRNA-based target gene expression inhibition system (pEKEx1-bsuhfq-roxS) constructed in Example 3. For this purpose, whether or not lysA production actually decreased in the BE strain capable of overproducing lysine, an amino acid, was tested, when targeting the lysA gene encoding diaminopimelate decarboxylase, the final step of lysine biosynthesis.

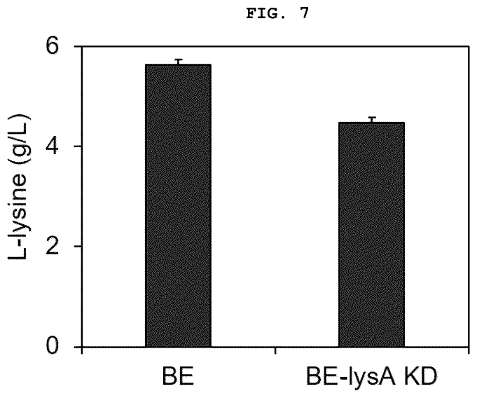

First, the sRNA targeting lysA was constructed on the pEKEx1-bsuhfq-roxS plasmid in the same manner as in Example 2, and was then transformed into a BE strain. The result of flask culture of the BE-lysA strain thus constructed along with the wild-type BE strain is shown in FIG. 7.

The flask culture conditions are as follows. The BE strain was inoculated in 5 mL of a BHIS medium (37 g/L of brain heart infusion (BHI), 91 g/L of sorbitol) and cultured at 30° C.; and 200 rpm for 18 hours. After 18 hours, the strain cultured in the BHIS medium was inoculated into a 300 mL baffle flask containing 25 mL of a LM medium (40 g/L of glucose, 1 g/L of K₂HPO₄, 1 g/L of KH₂PO₄, 1 g/L of urea, 20 g/L of (NH₄)₂SO₄, 10 g/L of yeast extract, 1 g/L of MgSO₄, 50 mg/L of CaCl₂, 0.1 mg/L of biotin, 10 mg/L of β-alanine, 10 mg/L of Thiamine-HCl, 10 mg/L of nicotinic acid, 5 mg/L of FeSO₄, 5 mg/L of MnSO₄, 2.5 mg/L of CuSO₄, 5 mg/L of ZnSO₄, 2.5 mg/L of NiCl₂, 1.5 g/L of CaCO₃) and cultured at 30° C. and 200 rpm for 24 hours. IPTG was added therewith when inoculating the strain.

As can be seen from FIG. 7, the production of lysine actually decreased by 20.7% due to inhibition of lysA expression by sRNA.

5-2. Confirmation of Pyc Gene Expression Inhibition Ability

As another example, a change in phenotype was observed when a pyc gene encoding pyruvate decarboxylase in the wild-type Corynebacterium glutamicum ATCC 13032 strain was used as a target for sRNA. The prior art literature reported that, when expression of the pyc gene was inhibited, the growth of C. glutamicum was significantly reduced in a medium containing sodium lactate as a carbon source (J. Park et al., Microb. Cell Fact. 2018, 17:4).

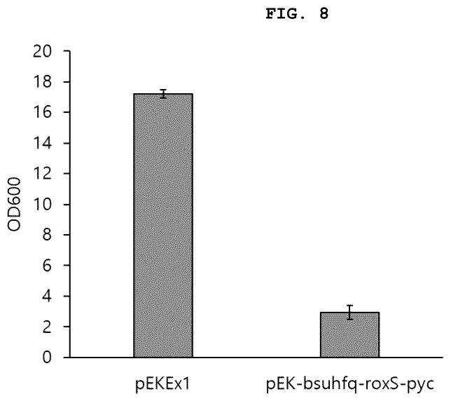

Accordingly, sRNA targeting pyc was constructed on the pEKEx1-bsuhfq-roxS plasmid and was then transformed into the C. glutamicum ATCC 13032 strain. The result of flask culture of the WT-pyc strain thus constructed along with the wild-type strain is shown in FIG. 8.

The flask culture conditions were as follows. The strain was inoculated in 5 mL of a BHIS medium (37 g/L of brain heart infusion (BHI), 91 g/L of sorbitol) and cultured at 30° C. and 200 rpm for 18 hours. After 18 hours, the strain cultured in the BHIS medium was inoculated into a 300 mL baffle flask containing 25 mL of a CGXII medium (20 g/L of (NH₄)₂SO₄, 5 g/L of urea, 1 g/L of KH₂PO₄, 1 g/L of K₂HPO₄, 0.25 g/L of MgSO₄·7H₂O, 42 g/L of 3-morpholinopropanesulfonic acid (MOPS), 13 mg/L of CaCl₂·2H₂O, 10 mg/L of FeSO₄·7H₂O, 14 mg/L of MnSO₄·5H₂O, 1 mg/L of ZnSO₄·7H₂O, 0.3 mg/L of CuSO₄·5H₂O, 0.02 mg/L of NiCl₂·6H₂O, 0.5 mg/L biotin, 30 mg/L of protocatechuic acid and 0.5 mg/L of thiamine) supplemented with 20 g/L of sodium lactate and cultured at 30° C. and 200 rpm for 24 hours. IPTG was added therewith when inoculating the strains.

As can be seen from FIG. 8, the growth of C. glutamicum in a medium containing sodium lactate as a carbon source actually decreased by 83% due to inhibition of pyc expression by sRNA. These results demonstrated that pEKEx1-bsuhfq-roxS, which is the synthetic regulatory sRNA platform constructed in the present disclosure, effectively inhibits the expression of a target gene in C. glutamicum.

### Example 6: Application of pEKEx1-Bsuhfq-roxS Synthetic Regulatory sRNA Platform to Different Strains

The construction of synthetic regulatory sRNA platforms capable of effectively acting in Gram-positive bacteria using Corynebacterium glutamicum as a representative sample of industrial Gram-positive bacteria has been disclosed in Examples 1 to 5. The present inventors further endeavored to prove that the synthetic regulated sRNA according to the present disclosure can be utilized as a universal tool that is effectively applicable to all industrial strains using E. coli, which is a representative industrial gram-negative bacterium, as a sample.

In order to confirm the inhibition ability on expression of the target gene in E. coli, the rppA gene of Streptomyces griseus was introduced, and this was used as the target gene. Type III polyketide biosynthetic enzymes expressed from the rppA gene produce a red pigment, called “flaviolin”, from malonyl-coA (Yang et al. (2018), Proc. Natl. Acad. Sci. U.S.A., 115(40): 9835-9844). Therefore, the present inventors tried to investigate the change in the production of flaviolin by inhibiting the expression of the rppA gene.

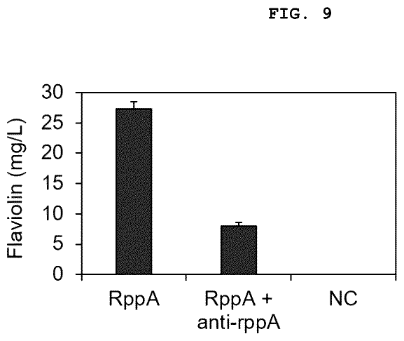

Accordingly, the pTacCDFS-5′UTR-Sgr_rppA plasmid was transformed into the E. coli BL21 (DE3) strain, a strain obtained by further transforming the pEKEx1 plasmid was prepared as a control strain, and a strain obtained by further transforming the rppA target sRNA plasmid was prepared as a strain to be tested. The results of measurement of the production of flaviolin after culturing the strains thus produced in LB medium are shown in FIG. 9.

As can be seen from FIG. 9, the production of flaviolin decreased by 70.8% through the introduction of pEKEx1-bsuhfq-roxS-based sRNA. Therefore, it can be seen that the sRNA system of the present disclosure can effectively inhibit the expression of a target gene in Gram-negative bacteria represented by E. coli.

E. coli is a representative industrially valuable Gram-negative bacterium and Corynebacterium is a representative industrially valuable Gram-positive bacterium. The present disclosure proved that the pEKEx1-bsuhfq-roxS system successfully acted in the two representative strains as described above, which suggests that the synthetic regulatory sRNA of the present disclosure is a general-purpose tool that is widely applicable to all kinds of microorganisms as disclosed in this example.

Although specific configurations of the present disclosure have been described in detail, those skilled in the art will appreciate that this description is provided to set forth preferred embodiments for illustrative purposes and should not be construed as limiting the scope of the present disclosure. Therefore, the substantial scope of the present disclosure is defined by the accompanying claims and equivalents thereto.

### INDUSTRIAL APPLICABILITY

The synthetic sRNA according to the present disclosure and the composition for inhibiting gene expression including the sRNA have the advantage of being capable of controlling single and multiple target genes at once, and the synthetic sRNA controlling gene expression is capable of effectively inhibiting expression of a target gene without the conventional gene deletion process and thus is useful for the production of recombinant microorganisms, particularly for inhibition of gene expression in Gram-positive bacteria. The recombinant Corynebacterium bacterium produced in the present disclosure is a recombinant microorganism that is capable of mass-producing high-value products based on a biological material in an environmentally friendly and renewable manner by regulating the microbial metabolic flow through synthetic sRNA. The recombinant microorganism, which is a biological material-based production system developed through such sRNA, can replace existing fossil fuels while solving environmental problems caused by the ever-increasing use of oils, and is thus useful.

What is claimed is:

1. A synthetic sRNA for inhibiting gene expression in a prokaryote, the synthetic sRNA comprising:
(i) an Hfq binding site of roxS sRNA comprising the sequence of SEQ ID NO: 42 derived from Bacillus subtilis amplified by a primer of SEQ ID NO: 19 or 20, or an Hfq binding site of arnA sRNA comprising the sequence of SEQ ID NO: 43 derived from Corynebacterium glutamicum amplified by a primer of SEQ ID NO: 15 or 16; and
(ii) a region forming a complementary bond with a target gene mRNA.

2. The synthetic sRNA according to claim 1, wherein the region forming the complementary bond with the target gene mRNA entirely or partially forms a complementary bond with nucleic acid sequences corresponding to a start of a ribosome binding site of the target gene mRNA to an end of a gene-coding sequence.

3. The synthetic sRNA according to claim 1, wherein the prokaryote is any one selected from the group consisting of Escherichia coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhizobium, Rhodobacter, Zymomonas, Bacillus, Staphylococcus, Lactococcus, Streptococcus, Lactobacillus, Clostridium, Corynebacterium, Streptomyces, Bifidobacterium and Cyclobacterium.

4. The synthetic sRNA according to claim 1, comprising the Hfq binding site of roxS sRNA comprising the sequence of SEQ ID NO: 42 derived from Bacillus subtilis amplified by a primer of SEQ ID NO: 19 or 20.

5. The synthetic sRNA according to claim 1, comprising the Hfq binding site of arnA sRNA comprising the sequence of SEQ ID NO: 43 derived from Corynebacterium glutamicum amplified by a primer of SEQ ID NO: 15 or 16.

6. A nucleic acid encoding the sRNA according to claim 1.

7. An expression vector comprising a nucleic acid encoding the sRNA according to claim 1.

8. A recombinant prokaryote transformed with the sRNA according to claim 1.

9. A recombinant prokaryote transformed with a nucleic acid encoding the sRNA according to claim 1.

10. A recombinant prokaryote transformed with the expression vector according to claim 7.

11. A nucleic acid comprising the nucleic acid according to claim 6 and a nucleic acid encoding prokaryote-derived Hfq.

12. The nucleic acid according to claim 11, wherein the prokaryote-derived Hfq is any one selected from the group consisting of Escherichia coli, Rhizobium, Bifidobacterium, Rhodococcus, Candida, Erwinia, Enterobacter, Pasteurella, Mannheimia, Actinobacillus, Aggregatibacter, Xanthomonas, Vibrio, Pseudomonas, Azotobacter, Acinetobacter, Ralstonia, Agrobacterium, Rhizobium, Rhodobacter, Zymomonas, Bacillus, Staphylococcus, Lactococcus, Streptococcus, Lactobacillus, Clostridium, Corynebacterium, Streptomyces, Bifidobacterium and Cyclobacterium.

13. A recombinant prokaryote transformed with the nucleic acid according to claim 11.

14. An expression vector comprising a nucleic acid encoding the sRNA according to claim 1 and a nucleic acid encoding prokaryote-derived Hfq.

15. A recombinant prokaryote transformed with the expression vector according to claim 14.

16. A method of inhibiting expression of a target gene in a prokaryote comprising culturing the recombinant prokaryote according to claim 15 to inhibit mRNA of the target gene.

17. A method of screening a gene targeted for deletion for production of a useful substance comprising:
(a) inhibiting expression of at least one of genes present in a target strain for producing the useful substance in a biosynthetic pathway of the useful substance, by the method according to claim 16; and
(b) selecting the gene, expression of which is inhibited, as the gene targeted for deletion for the production of the useful substance when a production yield of the useful substance is improved due to the inhibition of expression.

18. A method of improving a strain for producing a useful substance comprising deleting (i) a gene screened by the method according to claim 17, or (ii) a combination of genes including the screened gene, to produce a recombinant strain.


SEQUENCE LISTING (43 sequences)

SEQ ID NO: 1 - DNA, 47 bp, synthetic construct
  misc_feature 1..47: note = Synthetic construct
  source 1..47: mol_type = other DNA
caatttcacacaggaaacagaattcatggctaaggggcaatctttac

SEQ ID NO: 2 - DNA, 49 bp, synthetic construct
  misc_feature 1..49: note = Synthetic construct
  source 1..49: mol_type = other DNA
caccatatctatatctccttgaattcattattcggtttcttcgctgtcc

SEQ ID NO: 3 - DNA, 309 bp, synthetic construct
  misc_feature 1..309: note = Synthetic construct
  source 1..309: mol_type = other DNA
atggctaaggggcaatctttacaagatccgttcctgaacgcactgcgtcgggaacgtgttccagtttctatttatttggtgaatggtattaagctgcaagggcaaatcgagtcttttgatcagttcgtgatcctgttgaaaaacacggtcagccagatggtttacaagcacgcgatttctactgttgtcccgtctcgcccggtttctcatcacagtaacaacgccggtggcggtaccagcagtaactaccatcatggtagcagcgcgcagaatacttccgcgcaacaggacagcgaagaaaccgaataa

SEQ ID NO: 4 - DNA, 46 bp, synthetic construct
  misc_feature 1..46: note = Synthetic construct
  source 1..46: mol_type = other DNA
caatttcacacaggaaacagaattcatggctaagggtcagtctctc

SEQ ID NO: 5 - DNA, 47 bp, synthetic construct
  misc_feature 1..47: note = Synthetic construct
  source 1..47: mol_type = other DNA
caccatatctatatctccttgaattcattactattcggtttcctcgg

SEQ ID NO: 6 - DNA, 78 bp, synthetic construct
  misc_feature 1..78: note = Synthetic construct
  source 1..78: mol_type = other DNA
ttgacaattaatcatcggctcgtataatgtgtggagctctcattttgcagatttgttttagagctagaaatagcaagt

SEQ ID NO: 7 - DNA, 47 bp, synthetic construct
  misc_feature 1..47: note = Synthetic construct
  source 1..47: mol_type = other DNA
tatagatatcccgcggtatattaattaatataaacgcagaaaggccc

SEQ ID NO: 8 - DNA, 51 bp, synthetic construct
  misc_feature 1..51: note = Synthetic construct
  source 1..51: mol_type = other DNA
tggatgatggggcgattcaggtatagatatcttgacaattaatcatcggct

SEQ ID NO: 9 - DNA, 44 bp, synthetic construct
  misc_feature 1..44: note = Synthetic construct
  source 1..44: mol_type = other DNA
aaggtgttgctgactcataccaggtatagatatcccgcggtata

SEQ ID NO: 10 - DNA, 80 bp, synthetic construct
  misc_feature 1..80: note = Synthetic construct
  source 1..80: mol_type = other DNA
ttgacaattaatcatcggctcgtataatgtgtgggaaaagttcttctcctttactcattttctgttgggccattgcattg

SEQ ID NO: 11 - DNA, 46 bp, synthetic construct
  misc_feature 1..46: note = Synthetic construct
  source 1..46: mol_type = other DNA
caatttcacacaggaaacagaattcatgaaaccgattaatattcag

SEQ ID NO: 12 - DNA, 46 bp, synthetic construct
  misc_feature 1..46: note = Synthetic construct
  source 1..46: mol_type = other DNA
caaaacagccaagcttggctgcagattattcgagttcaagctggac

SEQ ID NO: 13 - DNA, 46 bp, synthetic construct
  misc_feature 1..46: note = Synthetic construct
  source 1..46: mol_type = other DNA
caatttcacacaggaaacagaattcatgattgcaaacgaaaacatc

SEQ ID NO: 14 - DNA, 42 bp, synthetic construct
  misc_feature 1..42: note = Synthetic construct
  source 1..42: mol_type = other DNA
caaaacagccaagcttggctgattattcttcactttcagtag

SEQ ID NO: 15 - DNA, 46 bp, synthetic construct
  misc_feature 1..46: note = Synthetic construct
  source 1..46: mol_type = other DNA
aaaggctattcaggggtaattttttcgggggatccactagttctag

SEQ ID NO: 16 - DNA, 50 bp, synthetic construct
  misc_feature 1..50: note = Synthetic construct
  source 1..50: mol_type = other DNA
aaaagactgttcgggggtaacgatgtctcgagccaggcatcaaataaaac

SEQ ID NO: 17 - DNA, 43 bp, synthetic construct
  misc_feature 1..43: note = Synthetic construct
  source 1..43: mol_type = other DNA
acccagtgacatgcttgggtgacgggggatccactagttctag

SEQ ID NO: 18 - DNA, 47 bp, synthetic construct
  misc_feature 1..47: note = Synthetic construct
  source 1..47: mol_type = other DNA
gttttttcttacgatagagagcactcgagccaggcatcaaataaaac

SEQ ID NO: 19 - DNA, 39 bp, synthetic construct
  misc_feature 1..39: note = Synthetic construct
  source 1..39: mol_type = other DNA
aagcgcggtttcatatgtcgggggatccactagttctag

SEQ ID NO: 20 - DNA, 43 bp, synthetic construct
  misc_feature 1..43: note = Synthetic construct
  source 1..43: mol_type = other DNA
atcccggcgcggtttctttctcgagccaggcatcaaataaaac

SEQ ID NO: 21 - DNA, 80 bp, synthetic construct
  misc_feature 1..80: note = Synthetic construct
  source 1..80: mol_type = other DNA
ttgacaattaatcatcggctcgtataatgtgtgggaaaagttcttctcctttactcataaaaaattacccctgaatagcc

SEQ ID NO: 22 - DNA, 78 bp, synthetic construct
  misc_feature 1..78: note = Synthetic construct
  source 1..78: mol_type = other DNA
ttgacaattaatcatcggctcgtataatgtgtgggaaaagttcttctcctttactcattcacccaagcatgtcactgg

SEQ ID NO: 23 - DNA, 79 bp, synthetic construct
  misc_feature 1..79: note = Synthetic construct
  source 1..79: mol_type = other DNA
ttgacaattaatcatcggctcgtataatgtgtgggaaaagttcttctcctttactcatacatatgaaaccgcgcttatc

SEQ ID NO: 24 - DNA, 20 bp, synthetic construct
  misc_feature 1..20: note = Synthetic construct
  source 1..20: mol_type = other DNA
tgcactactggaaaactacc

SEQ ID NO: 25 - DNA, 20 bp, synthetic construct
  misc_feature 1..20: note = Synthetic construct
  source 1..20: mol_type = other DNA
tgtagttcccgtcatctttg

SEQ ID NO: 26 - DNA, 19 bp, synthetic construct
  misc_feature 1..19: note = Synthetic construct
  source 1..19: mol_type = other DNA
gaaaacaccatcaccattc

SEQ ID NO: 27 - DNA, 20 bp, synthetic construct
  misc_feature 1..20: note = Synthetic construct
  source 1..20: mol_type = other DNA
gtctggtaaaccagggactc

SEQ ID NO: 28 - DNA, 312 bp, synthetic construct
  misc_feature 1..312: note = Synthetic construct
  source 1..312: mol_type = other DNA
atggctaagggtcagtctctccaggacccattcttgaacgcactgcgtcgcgaacgcgtgcccgtgtccatctatctggtgaacggtattaaacttcagggacagatcgagtccttcgatcagtttgttatcctgctcaagaacacggtctcccagatggtatacaagcatgcgatttcaaccgttgtcccttcccgcccggtgtctcaccactcgaacaatgccggcggcggcacctcctccaactaccaccacggcagcagcgcccaaaacacttccgcacagcaggattccgaggaaaccgaatagtaa

SEQ ID NO: 29 - DNA, 16 bp, synthetic construct
  misc_feature 1..16: note = Synthetic construct
  source 1..16: mol_type = other DNA
atgagcaaaggagaag

SEQ ID NO: 30 - DNA, 18 bp, synthetic construct
  misc_feature 1..18: note = Synthetic construct
  source 1..18: mol_type = other DNA
atgagcaaaggagaagaa

SEQ ID NO: 31 - DNA, 20 bp, synthetic construct
  misc_feature 1..20: note = Synthetic construct
  source 1..20: mol_type = other DNA
atgagcaaaggagaagaact

SEQ ID NO: 32 - DNA, 22 bp, synthetic construct
  misc_feature 1..22: note = Synthetic construct
  source 1..22: mol_type = other DNA
atgagcaaaggagaagaacttt

SEQ ID NO: 33 - DNA, 24 bp, synthetic construct
  misc_feature 1..24: note = Synthetic construct
  source 1..24: mol_type = other DNA
atgagcaaaggagaagaacttttc

SEQ ID NO: 34 - DNA, 26 bp, synthetic construct
  misc_feature 1..26: note = Synthetic construct
  source 1..26: mol_type = other DNA
atgagcaaaggagaagaacttttcac

SEQ ID NO: 35 - DNA, 28 bp, synthetic construct
  misc_feature 1..28: note = Synthetic construct
  source 1..28: mol_type = other DNA
atgagcaaaggagaagaacttttcactg

SEQ ID NO: 36 - DNA, 30 bp, synthetic construct
  misc_feature 1..30: note = Synthetic construct
  source 1..30: mol_type = other DNA
atgagcaaaggagaagaacttttcactgga

SEQ ID NO: 37 - DNA, 32 bp, synthetic construct
  misc_feature 1..32: note = Synthetic construct
  source 1..32: mol_type = other DNA
atgagcaaaggagaagaacttttcactggagt

SEQ ID NO: 38 - DNA, 34 bp, synthetic construct
  misc_feature 1..34: note = Synthetic construct
  source 1..34: mol_type = other DNA
atgagcaaaggagaagaacttttcactggagttg

SEQ ID NO: 39 - DNA, 36 bp, synthetic construct
  misc_feature 1..36: note = Synthetic construct
  source 1..36: mol_type = other DNA
atgagcaaaggagaagaacttttcactggagttgtc

SEQ ID NO: 40 - DNA, 38 bp, synthetic construct
  misc_feature 1..38: note = Synthetic construct
  source 1..38: mol_type = other DNA
atgagcaaaggagaagaacttttcactggagttgtccc

SEQ ID NO: 41 - DNA, 40 bp, synthetic construct
  misc_feature 1..40: note = Synthetic construct
  source 1..40: mol_type = other DNA
atgagcaaaggagaagaacttttcactggagttgtcccaa

SEQ ID NO: 42 - DNA, 37 bp, synthetic construct
  source 1..37: mol_type = other DNA
acatatgaaaccgcgcttatcccggcgcggtttcttt

SEQ ID NO: 43 - DNA, 51 bp, synthetic construct
  source 1..51: mol_type = other DNA
aaaaaattacccctgaatagcctttaaaagactgttcgggggtaacgatgt